(19) United States
(12) Patent Application Publication
(10) Pub. No.: US 2023/0080853 A1
(43) Pub. Date: Mar. 16, 2023

(54) METHOD FOR MANUFACTURING FIBER ASSEMBLY FOR PROVIDING BINDING SURFACE TO BIOSUBSTANCE AND FIBER ASSEMBLY, MANUFACTURED THEREBY, FOR PROVIDING BINDING SURFACE TO BIOSUBSTANCE

(71) Applicant: AMOGREENTECH CO., LTD., Gimpo-si, Gyeonggi-do (KR)

(72) Inventors: SeonHo JANG, Gimpo-si, Gyeonggi-do (KR); Hee Sung PARK, Gimpo-si, Gyeonggi-do (KR); Sung Il KIM, Gimpo-si, Gyeonggi-do (KR); Seam MUN, Gimpo-si, Gyeonggi-do (KR); Song Hee KOO, Gimpo-si, Gyeonggi-do (KR); Su Yeon LEE, Gimpo-si, Gyeonggi-do (KR); Ji Young KIM, Gimpo-si, Gyeonggi-do (KR); In Yong SEO, Gimpo-si, Gyeonggi-do (KR); Seoung Hoon LEE, Gimpo-si, Gyeonggi-do (KR); Chan KIM, Gimpo-si, Gyeonggi-do (KR)

(73) Assignee: AMOGREENTECH CO., LTD., Gimpo-si, Gyeonggi-do (KR)

(21) Appl. No.: 17/904,339

(22) PCT Filed: Feb. 17, 2021
(86) PCT No.: PCT/KR2021/002029

§ 371 Date: Aug. 16, 2022

(30) Foreign Application Priority Data
Feb. 17, 2020 (KR) 10-2020-0019067

(51) Int. Cl. C07K 17/08; C08L 79/02

(52) U.S. Cl. CPC C07K 17/08; C08L 79/02; C12N 11/089

(57) ABSTRACT

A method for manufacturing a fiber assembly for providing a binding surface to a bio-substance is provided. A fiber assembly for providing a binding surface to a bio-substance according to an embodiment of the present invention is manufactured by a method comprising the steps of: (1) preparing a fiber assembly in which a plurality of fibers is accumulated; and (2) performing modification to provide the fiber surface with a carboxyl group reactive to an amine group present in a bio-substance. According to the method, a bio-substance can be easily introduced at a high content into the fiber assembly. In addition, bio-substances that are conjugated therebetween and adsorbed through physical adsorption, etc. are remarkably reduced in the introduction procedure of bio-substances, whereby bio-substances can be availed with high precision and reliability in applications employing bio-substances. Furthermore, applications employing bio-substances can undergo minimal property variations attributed to the bio-substances as detachment of the bio-substances is minimized or prevented. Accordingly, the bio-substances fixed on the surface of the fiber assembly according to the present invention can find a broad spectrum of applications in various fields including the material engineering, bio engineering, medical fields, and so on.

### CROSS-REFERENCE TO RELATED APPLICATIONS

This application is a 35 U.S.C. 371 National Phase Entry Application from PCT/KR2021/002029 filed Feb. 17, 2021, which claims priority to and the benefit of Korean Patent Application No. 10-2020-0019067, filed on Feb. 17, 2020, the disclosures of which are incorporated herein by reference in their entirety.

### TECHNICAL FIELD

The present invention relates to a method for manufacturing a fiber assembly, and more particularly, to a method for manufacturing a fiber assembly for providing a binding surface to a bio-substance and a fiber assembly for providing a binding surface to a bio-substance manufactured by the method.

### BACKGROUND

Research on a technology for fixing various bio-substances to a structure is continuing, and the structure to which the bio-substance is fixed is widely used in various fields including material engineering, bio engineering, medical fields, and so on.

Conventionally, as a method for fixing bio-substances to structures, an ionic binding method using physical adsorption or a difference in surface charge has been widely used due to convenience in the process. However, such binding has a fatal disadvantage in that it cannot stably bind the bio-substance to the structure because the binding strength is weak and the bio-substance is easily detached from the structure.

Meanwhile, conventionally, beads have been open used as a structure for fixing bio-substances, and in particular, magnetic beads have been used in terms of ease of recovery after use. However, the magnetic beads do not have a large surface area, so there is a limit in the amount of bio-substances that can be fixed thereon.

In addition, a method of directly coating and fixing a bio-substance on the surface of a container as the structure in which the bio-substance is used, but when the bio-substance is simply coated, detachment of the bio-substance may be frequent. In addition, the coating may not be easy depending on the material of the container providing the surface on which the bio-substance is coated, and when the coating is performed on such material, there is a risk that the detachment of the bio-substance may be further accelerated. In addition, the detachment of bio-substances may reduce analytical sensitivity, accuracy, precision, stability, etc. in applications using bio-substances, for example, when detecting a target material using a bio-substance. Furthermore, if the bio-substances are clustered on the surface without a specific orientation and are randomly oriented, there is a risk that the binding efficiency or binding ability on the surface may be reduced due to steric hindrance. In addition, since the surface of the container can also contain the bio-substance only on the exposed surface area, there is a limit in increasing the content of the bio-substance.

Accordingly, there is an urgent need to study a structure having a binding surface capable of stably and highly integrating bio-substances.

### SUMMARY OF THE INVENTION

The present invention has been devised in view of the above matters, and an object of the present invention is to provide a method for manufacturing a fiber assembly for providing a binding surface to a bio-substance and a fiber assembly for providing a binding surface to a bio-substance prepared by the method, which are advantageous for fully expressing the function of the introduced bio-substances while preventing detachment as the bio-substance can be introduced in a larger amount and the introduced bio-substance is stably fixed.

In order to achieve the above object, the present invention provides a method for manufacturing a fiber assembly for providing a binding surface to a bio-substance, including the steps of: (1) preparing a fiber assembly in which a plurality of fibers is accumulated; and (2) performing modification to provide surfaces of the fibers with a carboxyl group reactive to an amine group present in a bio-substance.

According to an embodiment of the present invention, the fibers may include one type of polymer compound, a mixture of two or more compounds, or a copolymer obtained by copolymerizing two or more compounds selected from the group consisting of polyvinylidene fluoride, polyacrylonitrile, polyester, polyamide, polylactic acid, polyvinyl alcohol, polystyrene, polyglycolic acid, polyvinyl chloride, polyvinylpyrrolidone, polyurethane and polyethersulfone (PES).

In addition, the step (2) may include the steps of 2-1) treating a surface of the fiber assembly with an alkali solution of pH 12.0 or higher, and 2-2) treating the surface of the fiber assembly treated with the alkali solution with a solution containing a carboxyl group-containing compound.

In addition, the carboxyl group-containing compound may include a compound having at least three or more carboxyl groups. In this case, the carboxyl group-containing compound may be citric acid.

In addition, the fiber assembly may include polyacrylonitrile fiber, and the carboxyl group-containing compound may be citric acid.

In addition, the steps 2-1) and 2-2) may be each independently performed at a temperature of 50 to 70° C. for 24 hours or more.

In addition, the solution containing the carboxyl group-containing compound may include at least one of alcohol and water as a solvent, the alcohol may be at least one selected from the group consisting of alcohols having 3 to 10 carbon atoms. In this case, as an example, the solvent may include water and alcohol in a weight ratio of 1:0.2 to 1.8.

In addition, the solution containing the carboxyl group-containing compound may contain the compound containing the carboxyl group at a concentration of 8 to 15M.

In addition, the present invention provides a fiber assembly for providing a binding surface to a bio-substance, which is formed of a plurality of fibers in which a carboxy group reactive to an amino group present in the bio-substance is provided on a surface according to the method of the present invention.

According to an embodiment of the present invention, the fibers may include polyacrylonitrile fibers, and the carboxyl group may be derived from citric acid.

In addition, the fibers may have an average diameter of less than 1 μm.

In addition, the present invention provides a fiber assembly to which a bio-substance is fixed, including the fiber assembly according to the present invention; and the bio-substance including at least one amine group, wherein the amine group forms a covalent bond with the carboxyl group provided on surfaces of the fibers in the fiber assembly and is bonded to the surfaces of the fibers.

According to an embodiment of the present invention, the bio-substance may include any one or more of an enzyme, a biosignal molecule, and a biomolecule that has at least one amine group or is modified to have at least one amine group, the enzyme may include one or more selected from the group consisting of carbonic anhydrase, glucose oxidase, trypsin, chymotrypsin, subtilisin, papain, thermolysin, lipase, peroxidase, acylase, lactonase, protease, tyrosinase, laccase, cellulase, xylanase, organophosphohydrolase, cholinesterase, formate dehydrogenase, aldehyde dehydrogenase, alcohol dehydrogenase, glucose dehydrogenase and glucose isomerase, the biosignal molecule may include one or more selected from the group consisting of chemokine, cytokine, cell survival factor, cell proliferation factor, and cell differentiation factor, the biomolecule may include one or more selected from the group consisting of albumin, insulin, collagen, antibody, antigen, protein A, protein G, avidin, streptavidin, neutravidin, biotin, nucleic acid, peptide, lectin, glycosyl protein, cells and carbohydrates. The method for manufacturing a fiber assembly for providing a binding surface to a bio-substance according to the present invention can easily introduce a bio-substance into the fiber assembly in a high content. In addition, bio-substances that are conjugated therebetween and adsorbed through physical adsorption, etc. are remarkably reduced in the introduction procedure of bio-substances, whereby bio-substances can be availed with high precision and reliability in applications employing bio-substances. In addition, applications employing bio-substances can undergo minimal property variations attributed to the bio-substances as detachment of the bio-substances is minimized or prevented. Accordingly, the bio-substances fixed on the surface of the fiber assembly according to the present invention can find a broad spectrum of applications in various fields including the material engineering, bio engineering, medical fields, and so on.

### BRIEF DESCRIPTION OF THE DRAWINGS

FIG. 1 shows an FTIR spectrum for Example and Comparative Example of the present invention.

### DETAILED DESCRIPTION

Hereinafter, embodiments of the present invention will be described in detail so that those of ordinary skill in the art to which the present invention pertains can easily implement them. The present invention may be embodied in many different forms and is not limited to the embodiments described herein.

The method for manufacturing a fiber assembly for providing a binding surface to a bio-substance according to the present invention includes the steps of (1) preparing a fiber assembly in which a plurality of fibers is accumulated; and (2) performing modification to provide surfaces of the fibers with a carboxyl group reactive to an amine group present in a bio-substance.

First, as the step (1), the step of preparing a fiber assembly in which a plurality of fibers is accumulated is performed.

The fiber assembly formed of fibers functions as a support for supporting the bio-substance to be introduced. The fiber assembly may be formed by randomly accumulating the plurality of fibers in a height direction, and thus may have a three-dimensional network structure and a non-uniform surface morphology. The surface of the fiber assembly is substantially the assembly of the outer surfaces of the respective fibers, and thus, there is an advantage in that the specific surface area that can be provided with the bio-substance can be significantly increased compared to the non-porous support of the same volume.

The fiber assembly may be an article commonly referred to as a fiber web, and the method for manufacturing the fiber assembly may employ a conventional method for manufacturing a fiber web, so the present invention is not particularly limited with respect to a specific method for preparing the fiber assembly.

However, in order to realize a more improved specific surface area, the fiber assembly according to an embodiment of the present invention may be formed of fibers having an average diameter of 2 μm or less, more preferably less than 1 μm. The fibers having such a small diameter may be manufactured according to a commonly used manufacturing method of microfibers manufactured through a reduction process after melt-spun into sea-island yarns, or may be manufactured using electrospinning. When electrospinning is used, the fiber assembly can be formed directly on the surface of the receiving space in the container on which the bio-substance is to be supported through spinning, so that the process of attaching the support to the container is omitted. Thus, there is the advantage of simplifying the manufacturing process and shortening the manufacturing time.

The electrospinning may be performed by adopting a known electrospinning apparatus and general electrospinning conditions as it is or by changing them appropriately. Specifically, considering the material and content of the fiber-forming component contained in the spinning solution to be spun, the type of solvent that dissolves it, and the diameter of the desired fiber, the strength of the voltage applied during the electrospinning, the height of an air gap, and the humidity and temperature in the air gap, etc. can be appropriately changed.

In addition, the fiber-forming component forming the fiber may be appropriately selected from known fiber-forming components in consideration of the manufacturing method of the fiber and desired physical properties such as chemical resistance, mechanical strength, and flexibility. For example, the fiber-forming component may include one type of polymer compound, a mixture of two or more compounds, or a copolymer obtained by copolymerizing two or more compounds selected from the group consisting of polyvinylidene fluoride, polyacrylonitrile, polyester, polyamide, polylactic acid, polyvinyl alcohol, polystyrene, polyglycolic acid, polyvinyl chloride, polyvinylpyrrolidone, polyurethane and polyethersulfone (PES). Preferably, the fiber-forming component may be polyacrylonitrile, and in this case, there is an advantage in that the fiber assembly can be provided with a bio-substance to be described later in an increased content, compared to other types of fiber-forming components.

In addition, the basis weight of the fiber assembly may be, for example, 1 to 100 g/m², and may be appropriately adjusted according to the purpose.

Next, as the step (2) according to the present invention, the step of modifying the fiber assembly so that a carboxyl group is provided on the surfaces of the fibers is performed.

The carboxyl group is a binding functional group for fixing the bio-substance to the fiber assembly, and more specifically, it is provided as a functional group for forming an amide bond with an amine group present in the bio-substance.

The carboxyl group may be provided on the surface of the fiber through a known modification method. Preferably, the carboxy group may be provided on the surface of the fiber by the step including the step 2-1) of treating the surface of the fiber assembly with an alkali solution, the step 2-2) of treating the surface of the fiber assembly treated with the alkali solution with a solution containing a carboxyl group-containing compound.

The step 2-1) is the step of contacting the surface of the fiber assembly, specifically, the surfaces of the fibers with the alkali solution, through which a hydroxyl group can be formed on the fiber surface. The alkali solution may be, for example, sodium hydroxide, wherein the concentration of the alkaline solution may be 1 to 20 M, preferably to 10 M. In addition, the alkali solution may have a pH of 12.0 or more, or 13.0 or more, for example, 13.5. In addition, a specific method of treating the alkali solution may be, for example, impregnation. In addition, the treatment temperature of the alkali solution may be 50 to 70° C. In addition, the treatment time of the alkali solution may be 1 minute to 30 hours, more preferably 15 hours or more, even more preferably 24 hours or more. If the treatment time of the alkali solution is not sufficient, it may be difficult to achieve a significant increase in the amount of bio-substance introduced into the fiber assembly.

Thereafter, as the step 2-2), the step of treating the surface of the fiber assembly treated with the alkali solution with the solution containing a carboxyl group-containing compound may be performed. In this case, the water washing process for the fiber assembly treated with the alkali solution may be further performed before performing the step 2-2), but is not limited thereto.

The carboxyl group-containing compound may be a compound having at least three or more carboxyl groups. For example, the carboxyl group-containing compound may be one or more selected from the group consisting of citric acid, poly(acrylic acid-co-maleic acid), polyacrylic acid, poly(styrenesulfonic acid-co-maleic acid) and poly(styrene-co-maleic acid). If the carboxyl group-containing compound has two carboxyl groups, there is a risk that the amount of bio-substance introduced into the fiber assembly may be significantly reduced.

The carboxyl group-containing compound may preferably be citric acid. In this case, there are advantages of increasing the introduction amount of the bio-substance compared to the case of using a polymer-type compound such as polyacrylic acid, preventing non-specific introduction of the bio-substance, and fully expressing the function of the introduced bio-substance.

The solution containing the carboxyl group-containing compound may contain as a solvent any one of an organic solvent and water. The organic solvent may be one or more selected from the group consisting of a straight-chain or pulverized alcohol having to 10 carbon atoms, more preferably 3 to 10 carbon atoms. More preferably, the solvent may further include any one or more of propanol and isopropyl alcohol, and in this case, there is the advantage of further increasing the amount of carboxyl group introduced.

In addition, preferably, the solvent may contain water and alcohol in a weight ratio of 1:0.2 to 1.8. In this case, the vaporization of the solution is minimized even when the reaction time of step 2-2) is prolonged, so that there is advantage in that the carboxyl group is more stably provided on the fiber surface. In addition, the concentration of the carboxyl group-containing compound in the solution may be 1 to 20 M, preferably 8 to 15 M. in this case, there is the advantage in that the carboxyl group is provided in a sufficient amount and at the same time the function of the bio-substance introduced through this can be fully expressed. If the concentration of the carboxyl group-containing compound in the solution is out of the preferred range and contained in at a high concentration, the increase in the amount of the carboxyl group may be insignificant. If the bio-substance has several amine groups, there is a risk that the bio-substance may be denatured or reduced in function as one bio-substance and several carboxyl groups are combined.

In addition, the step 2-2) may be performed for 1 minute to 30 hours, more preferably for 15 hours or more, and even more preferably for 24 hours or more, at a temperature of 50 to 70° C. If the treatment and reaction time of the solution containing the carboxyl group-containing compound is insufficient, it may be difficult to achieve a significant increase in the amount of the bio-substance introduced into the fiber assembly.

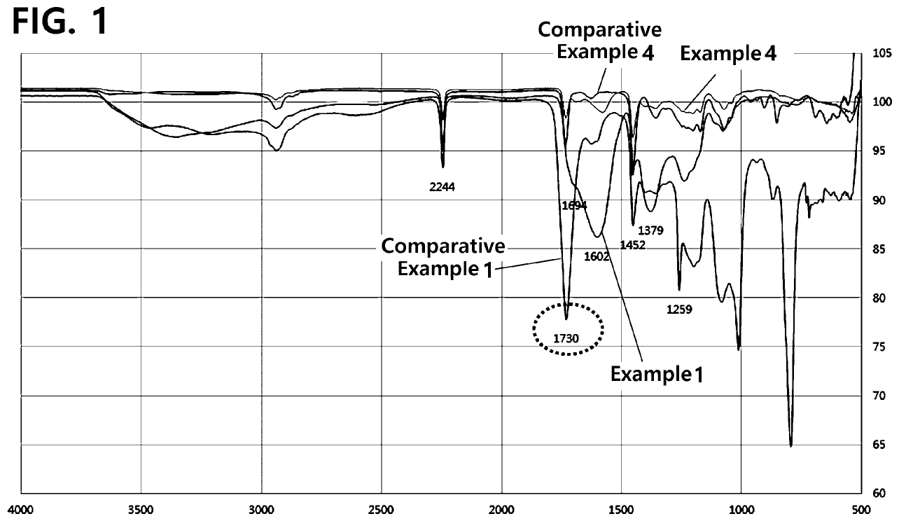

On the other hand, the present invention uses a method of modifying the surface of the fiber so that the carboxyl group is exposed to the surface. Compared to the case in which the compound having a carboxyl group is included in the spinning solution and is spun together, there is the advantage of significantly increasing the amount of bio-substance bound to the fiber assembly. This is a very specific effect that is different from the results expected from the FT-IR data for the fiber assembly provided with the carboxyl group. Specifically, with reference to FIG. 1, it can be seen that the peak at 1740 cm-1 corresponding to the carboxyl group in Comparative Example corresponding the fiber assembly manufactured by including the compound containing the carboxyl group in the spinning solution and spinning them together is clear and large compared to Example 1 in which the fiber assembly was treated with the alkali solution and then treated with the solution containing the compound containing the carboxyl group to modify the surface. However, as can be seen in Table 1 to be described later, in the amount of biotin corresponding to the introduced amount of streptavidin, which is an example of a bio-substance, Comparative Example 1 introduces only avidin at a level less than 1/100 of that of Example 1. This is contrary to what is expected from the FT-IR data, and it can be seen that the fiber assembly according to the present invention has a binding ability to introduce a very large amount of bio-substances.

In addition, the present invention can implement the fiber assembly for providing the binding surface to the bio-substance, which is formed of the plurality of fibers in which the carboxy group reactive to the amino group present in the bio-substance is provided on the surface, through the above described manufacturing method.

In this case, preferably, the fibers include polyacrylonitrile fibers, and the carboxyl group may be derived from citric acid, and in this case, there is an advantage in that the bio-substance can be introduced in a significantly increased amount into the fiber assembly.

In addition, the present invention includes a fiber assembly to which a bio-substance is fixed, including the fiber assembly according to the present invention, and the bio-substance including at least one amine group, wherein the amine group forms a covalent bond with the carboxyl group provided on surfaces of the fibers in the fiber assembly and is bonded to the surfaces of the fibers.

The bio-substance is a known material used in the fields of material engineering, bio engineering, or medical fields, and it may include a material that exists in an organism or a material that can be used for a biological reaction even if it is not a material that exists in an organism. The bio-substance may be an organic material or an inorganic material, and in the case of an organic material, for example, it may be in the form of carbohydrates, proteins, nucleic acids, lipids, or small molecules forming them. The bio-substance may include any one or more of an enzyme, a biosignal molecule, and a biomolecule that has at least one amine group or is modified to have at least one amine group.

As the enzyme, a known enzyme can be used without limitation. The example may include one or more selected from the group consisting of carbonic anhydrase, glucose oxidase, trypsin, chymotrypsin, subtilisin, papain, thermolysin, lipase, peroxidase, acylase, lactonase, protease, tyrosinase, laccase, cellulase, xylanase, organophosphohydrolase, cholinesterase, formate dehydrogenase, aldehyde dehydrogenase, alcohol dehydrogenase, glucose dehydrogenase and glucose isomerase.

In addition, as the biosignal molecule, any known biosignal molecule may be used without limitation, and the example may include one or more selected from the group consisting of chemokine, cytokine, cell survival factor, cell proliferation factor, and cell differentiation factor.

In addition, the biomolecule may include one or more selected from the group consisting of albumin, insulin, collagen, antibody, antigen, protein A, protein G, avidin, streptavidin, neutravidin, biotin, nucleic acid, peptide, lectin, glycosyl protein, cells and carbohydrates.

In addition, the bio-substance may further include a separate labeling material for identifying the bio-substance, and the labeling material may be a fluorescent material, a fluorescent material-binding protein, a luminescent material, a luminescent material-binding protein, or an enzyme. Since an appropriate known labeling substance can be selected, the present invention is not particularly limited thereto.

In addition, the bio-substance may be bound to the carboxyl group on the fiber assembly through a known method, and may be introduced into the fiber surface through, for example, EDC or EDC/NHS coupling reaction.

The fiber assembly having the binding surface for bio-substances according to the present invention can be applied to various applications such as various kits, devices, and biosensors used for detection, diagnosis, and analysis, supports for cell or tissue culture, and bio-cells. Thus, the fiber assembly can be widely used throughout the industry.

### EXAMPLES

The present invention will be described in more detail through the following examples, but the following examples are not intended to limit the scope of the present invention, which should be construed to aid understanding of the present invention.

### Example 1

A polyacrylonitrile (PAN) nanofiber web having the average diameter of 300 μm and the basis weight of 30 g/m² was prepared. The prepared nanofiber web was impregnated with an aqueous solution of sodium hydroxide at a concentration of 5M and 60° C. and treated with an alkali solution for 24 hours. Then, the alkali solution-treated nanofiber web was impregnated in a solution containing citric acid at a concentration of 10 M in a propanol solvent and reacted at 60° C. for 24 hours to prepare a fiber assembly having a carboxyl group on the fiber surface.

### Example 2

Except that the fiber assembly was changed to the polyvinylidene fluoride nanofiber web having the average diameter of 500 μm and the basis weight of 30 g/m², the fiber assembly having a carboxyl group provided on the fiber surface was prepared in the same manner as in Example 1.

### Example 3

Except that the fiber assembly was changed to the polyurethane/polyvinylidene fluoride nanofiber web having the average diameter of 600 μm and the basis weight of 30 g/m², the fiber assembly having a carboxyl group provided on the fiber surface was prepared in the same manner as in Example 1. In this case, the nanofiber web was prepared through a spinning solution in which polyurethane and polyvinylidene fluoride were mixed in a weight ratio of 5:5.

### Comparative Example 1

For preparing a spinning solution, first a mixed solution was prepared by dissolving 36 g of polyacrylonitrile in dimethylacetamide/acetone 114.8 g/49.2 g at a temperature of 80° C. for 6 hours using a magnetic bar. Then, after cooling the mixed solution to room temperature, 1.8 g of citric acid was mixed with 200 mL of the mixed solution to prepare the spinning solution. The prepared spinning solution was put into the solution tank of an electrospinning device, and discharged at a rate of 15 μl/min/hole to prepare the fiber assembly that was the nanofiber web having the average diameter of 300 μm and the basis weight of 25 g/m². In this case, the temperature of the spinning section was 28° C. and the humidity was maintained at 40%, and the distance between a collector and a spinning nozzle tip was 18 cm. Thereafter, the prepared fiber assembly was treated with the solution containing an alkali solution in the same manner as in Example 1 to prepare the fiber assembly having the carboxyl group on the fiber surface.

### Comparative Example 2

Except that polyacrylonitrile in the spinning solution was changed to polyvinylidene fluoride, the fiber assembly that was the nanofiber web having the average diameter of 500 μm and the basis weight of 30 g/m² was prepared in the same manner as in Comparative Example 1. Therefore, the fiber assembly in which the carboxy group is provided on the fiber surface was prepared.

### Comparative Example 3

Except that polyacrylonitrile was changed to polyurethane and polyvinylidene fluoride having the weight ratio of 3:7 in the spinning solution, the fiber assembly that was the nanofiber web having the average diameter 500 μm and the basis weight 30 g/m² was prepared in the same manner as in Comparative Example 1. Therefore, the fiber assembly in which the carboxy group is provided on the fiber surface was prepared.

### Experimental Example 1

According to Examples 1 to 3 and Comparative Examples 1 to 3, the fiber assemblies in which the carboxyl groups were provided on the fiber surface were prepared as specimens having the length and width of 1 cm and 5 cm, respectively. After the specimens were impregnated in 2.5 ml of pH 6, 15 mM MES, 25 mg/2.5 ml EDC (in MES) was mixed and reacted for about 30 minutes. Then, the supernatant was removed, and 4 mg StAv/5 ml (in MES) was added to introduce the streptavidin (StAv) into the fiber assemblies. Thereafter, the elution was performed using an elution buffer of pH 2, washed 3 times with 5 ml of PBST, and then the binding ability was evaluated using FITC-Biotin (Thermo Fisher) according to the manufacturer's manual and protocol. The results were shown in Table 1 below.

TABLE 1

| | | | | Comparative | Comparative | Comparative |
|---|---|---|---|---|---|---|
| | Example 1 | Example 2 | Example 3 | Example 1 | Example 2 | Example 3 |
| Material of | PAN | PVDF | PU/PVDF | PAN | PVDF | PU/PVDF |
| fiber | | | | | | |
| assembly | | | | | | |
| Content of | 7,916 | 691 | 1,189 | 76 | 35 | 375 |
| Free biotin | | | | | | |
| (pmol) | | | | | | |

As can be seen from Table 1, it can be found that the fiber assemblies according to Examples have the ability to significantly introduce bio-substances compared to the assemblies according to Comparative Examples in which the carboxyl group-containing compound was included in the spinning solution to provide the carboxyl group on the surface.

### Example 4

The fiber assembly was prepared in the same manner as in Example 1, except that the treatment time of the alkali solution was changed to 1 minute to prepare the fiber assembly having the carboxyl group on the fiber surface.

### Comparative Example 4

The same polyacrylonitrile (PAN) nanofiber web as in Example 1 was prepared, but the modification treatment was not performed.

### Experimental Example 2

FT-IR spectra of the fiber assemblies according to Examples 1, 4, Comparative Example 1, and Comparative Example 4 were identified using a Raman spectrometer (LabRam ARAMIS IR2), and the results were shown in FIG. 1 below.

As can be seen in FIG. 1, it was found that the peak at 1740 cm⁻¹ corresponding to the carboxy group in the fiber assembly of Comparative Example 1 in which the compound containing the carboxyl group was contained in the spinning solution and was spun together was clear and large, compared to the peak in the fiber assembly of Example 1 in which the fiber assembly was treated with the alkali solution and then was modified by treating with the solution containing the compound containing the carboxyl group.

However, as can be seen in Table 1, in the amount of biotin corresponding to the introduction amount of avidin which is an example of a bio-substance, it was found that in Comparative Example 1, streptavidin was introduced at a level less than 1/100 as in Example 1. This result is contrary to what is expected from the FT-IR data, and it can be seen that the fiber assembly according to the present invention has a binding ability to introduce a very large amount of bio-substance.

### Example 5

The fiber assembly having the carboxyl group provided on the fiber surface was prepared in the same manner as in Example 1, except that the fiber assembly was changed to the polyacrylonitrile (PAN) nanofiber web having the average diameter of 300 μm and the basis weight of 30 g/m², the concentration of the alkali solution was changed to the sodium hydroxide at 10M concentration, and the treatment time of the alkali solution and the solution containing the carboxyl group-containing compound was changed to 8 hours.

### Example 6

The fiber assembly having the carboxyl group provided on the fiber surface was prepared in the same manner as in Example 1, except that the fiber assembly was changed to the polyacrylonitrile (PAN) nanofiber web having the average diameter of 300 μm and the basis weight of 30 g/m², the concentration of the alkali solution was changed to the sodium hydroxide at 10M concentration, and the treatment time of the alkali solution and the solution containing the carboxyl group-containing compound was changed to 24 hours.

### Experimental Example 3

The fiber assembly to which streptavidin was fixed was prepared in the same manner as in Experimental Example 1, and the binding ability was evaluated in the same way, and the results were shown in Table 2 below.

TABLE 2

| | Example 5 | Example 6 |
|---|---|---|
| Treatment time of alkali solution/Treatment time | 8 hours/ | 24 hours/ |
| of a solution containing a carboxyl group- | 8 hours | 24 hours |
| containing compound | | |
| Content of free biotin (pmol) | 3,463 | 33,450 |
| Free biotin content per treatment time (pmol/ | 432.9 | 1393.8 |
| treatment time) | | |

As can be seen from Table 2, it was found that Example 6, in which the alkali solution and the solution containing the carboxyl group-containing compound were treated for 24 hours, exhibited a significantly larger amount of introduced bio-substance than that of Example 5. Also, it was found through the amount introduced per treatment time that Example 6 manufactured the fiber aggregate capable of achieving a remarkable binding amount three times or more of the level predicted in Example 5.

Although one embodiment of the present invention has been described above, the spirit of the present invention is not limited to the embodiments presented herein. Those skilled in the art who understand the spirit of the present invention will be able to easily suggest other embodiments by adding, changing, deleting, or including components within the scope of the same spirit, and this will also fall within the scope of the present invention.

1. A method for manufacturing a fiber assembly for providing a binding surface to a bio-substance, comprising the steps of:
(1) preparing a fiber assembly in which a plurality of fibers is accumulated; and
(2) performing modification to provide surfaces of the fibers with a carboxyl group reactive to an amine group present in a bio-substance.

2. The method according to claim 1, wherein the fibers include one type of polymer compound, a mixture of two or more compounds, or a copolymer obtained by copolymerizing two or more compounds selected from the group consisting of polyvinylidene fluoride, polyacrylonitrile, polyester, polyamide, polylactic acid, polyvinyl alcohol, polystyrene, polyethylene, polyglycolic acid, polyvinyl chloride, polyvinylpyrrolidone, polyurethane and polyethersulfone (PES).

3. The method according to claim 1, wherein the step (2) includes the steps of:
2-1) treating a surface of the fiber assembly with an alkali solution of pH 12.0 or higher; and
2-2) treating the surface of the fiber assembly treated with the alkali solution with a solution containing a carboxyl group-containing compound to provide the carboxy group on the surfaces of the fibers.

4. The method according to claim 3, wherein the carboxyl group-containing compound is a compound having at least three or more carboxyl groups.

5. The method according to claim 3, wherein the fiber assembly includes polyacrylonitrile fiber, and the carboxyl group-containing compound is citric acid.

6. The method according to claim 3, wherein the steps 2-1) and 2-2) are each independently performed at a temperature of 50 to 70° C. for 24 hours or more.

7. The method according to claim 3, wherein the solution containing the carboxyl group-containing compound includes at least one of alcohol and water as a solvent, the alcohol is at least one selected from the group consisting of alcohols having to 10 carbon atoms.

8. The method according to claim 3, wherein the solution containing the carboxyl group-containing compound contains the compound containing the carboxyl group at a concentration of 8 to 15M.

9. A fiber assembly for providing a binding surface to a bio-substance, which is formed of a plurality of fibers in which a carboxy group reactive to an amino group present in the bio-substance is provided on a surface according to the method of claim 1.

10. The fiber assembly according to claim 9, wherein the fibers include polyacrylonitrile fibers, and the carboxyl group is derived from citric acid.

11. The fiber assembly according to claim 9, wherein the fibers have an average diameter of less than 1 μm.

12. A fiber assembly to which a bio-substance is fixed, comprising:
the fiber assembly according to claim 9; and
the bio-substance including at least one amine group, wherein the amine group forms a covalent bond with the carboxyl group provided on surfaces of the fibers in the fiber assembly and is bonded to the surfaces of the fibers.

13. The fiber assembly according to claim 12, wherein the bio-substance includes any one or more of an enzyme, a biosignal molecule, and a biomolecule that has at least one amine group or is modified to have at least one amine group.
the enzyme includes one or more selected from the group consisting of carbonic anhydrase, glucose oxidase, trypsin, chymotrypsin, subtilisin, papain, thermolysin, lipase, peroxidase, acylase, lactonase, protease, tyrosinase, laccase, cellulase, xylanase, organophosphohydrolase, cholinesterase, formate dehydrogenase, aldehyde dehydrogenase, alcohol dehydrogenase, glucose dehydrogenase and glucose isomerase,
the biosignal molecule includes one or more selected from the group consisting of chemokine, cytokine, cell survival factor, cell proliferation factor, and cell differentiation factor,
the biomolecule includes one or more selected from the group consisting of albumin, insulin, collagen, antibody, antigen, protein A, protein G, avidin, streptavidin, neutravidin, biotin, nucleic acid, peptide, lectin, glycosyl protein, cells and carbohydrates.
